(19) United States
(12) Patent Application Publication
(10) Pub. No.: US 2014/0046202 A1
(43) Pub. Date: Feb. 13, 2014

(54) NONINVASIVE SYSTEMS FOR AORTIC ANEURYSM EVALUATION

(71) Applicants: Niema Pahlevan, Pasadena, CA (US); Morteza Gharib, Altadena, CA (US)

(72) Inventors: Niema Pahlevan, Pasadena, CA (US); Morteza Gharib, Altadena, CA (US)

(73) Assignee: CALIFORNIA INSTITUTE OF TECHNOLOGY, Pasadena, CA (US)

(21) Appl. No.: 13/964,631

(22) Filed: Aug. 12, 2013

Related U.S. Application Data
(60) Provisional application No. 61/682,523, filed on Aug. 13, 2012.

(51) Int. Cl. A61B 5/02; A61B 5/0265; A61B 5/00; A61B 8/06

(52) U.S. Cl. CPC A61B 5/02014; A61B 8/06; A61B 5/0265; A61B 5/7278; A61B 5/7246; A61B 5/7275
USPC 600/504

(57) ABSTRACT

Hardware and software methodology are described for non-invasive approaches to aortic aneurysm evaluation using ultrasound, microwave, and/or other radiofrequency (RF) techniques. Embodiments can be used to diagnose AAA and other aortic aneurysm conditions by non-invasive measurement and computation of displacement-based wave intensity (DWI) and/or displacement-based reflected wave intensity (DRWI) along the aorta and by comparison of the results to baseline data for a given patent or a population sample catalogue. Deviation of DWI and/or DRWI from a normal condition can be used to assess the severity of the AAA or other aortic aneurysm and any associated rupture risk.

### RELATED APPLICATIONS

This application claims the benefit of and priority to U.S. Provisional Patent Application Ser. No. 61/682,523, filed Aug. 13, 2012, which is incorporated by reference herein in its entirety for all purposes.

### FIELD

This subject matter relates to non-invasive diagnosis of aortic aneurysms and assessments of their risk of rupture.

### BACKGROUND

Aortic aneurysms involve dilation of a section of aorta. Aortic aneurysms can be categorized based on their location (aortic root aneurysm, thoracic aortic aneurysm, thorcoabdominal aortic aneurysm and abdominal aortic aneurysm), their shape (fusifrom and saccular) or their underlying cause. Among these, abdominal aortic aneurysms (AAA) are the most common type. AAAs cause more than 13,000 deaths per year in US alone.

Currently, there are approximately 15,000 patients each year diagnosed with an AAA and the number is growing every year. Early stage AAAs produce no or few symptoms. They are usually diagnosed accidentally when ultrasound or CT-scan procedures are performed for other purposes. If an AAA ruptures, the mortality rate is 65% to 85%.

Aneurysm size is the most important factor in predicting the likelihood of rupture. However, size is not a perfect predictor of rupture as there are some small aneurysms that rupture and there are some large aneurysms that do not. Two types of AAAs have been proposed based on rupture risk. Type I AAAs are those for which enlargement is accompanied by increasing wall stiffness and, hence, the risk rupture is low. Type II AAAs are those for which wall stiffness does not increase as the aneurysm size grows.

Wave dynamics in a compliant tube is a complex phenomenon that depends on fundamental frequency of the propagating waves, compliance of the tube, and reflection sites. The heart is a pulsatile pumping system and the aorta is the largest and most compliant vessel that extends from the heart. Therefore, aortic wave dynamics have a significant influence on arterial waves. Aortic wave dynamics depend on heart rate (HR), aortic compliance (AC), and reflection sites, to name a few.

Reflection sites can be categorized based on their overall function as closed-end reflection sites (CRS) or open-end reflection sites (ORS). In CRS, pressure waves are reflected positively and flow waves are reflected negatively. Conversely, in ORS, pressure waves are reflected negatively and flow waves are reflected positively.

In the aorta and systemic arterial system, all reflection sites are CRS. However, aortic aneurysms act like an ORS since diameter increases and, in most of cases, compliance as well. This additional ORS changes the wave dynamics in the aorta and its major branches.

Such an understanding of a study of AAA effect on wave reflection in the aorta is treated by Swillens, et al. at IEEE Transactions on Biomedical Engineering, Vol. 55. No. 5, May 2008. As described, a silicon 3D model reconstruction was made of a 78 year old male having an AAA. Pressure and flow waves were measured at different locations within the model characterizing it before and after simulated repair. Numerical modeling that was also presented offered general agreement with the testing.

Lacking, however, is a practical application of such knowledge. Especially one for non-contacting and/or non-invasive techniques of AAA evaluation.

### SUMMARY

The embodiments described herein are directed to systems, devices, and methods that provide an approach for AAA evaluation performed, in some embodiments, non-invasively using ultrasound and/or microwave or other radiofrequency (RF) techniques. The embodiments of these systems and devices include sensor hardware and computer processors and other ancillary/support electronics and various housing elements. The embodiments of the methods include the hardware and software for carrying out the same. Non-invasive AAA evaluation can be performed without reliance on an assumption of consistent vessel wall elasticity that may not hold as discussed above regarding Type I and Type II AAAs. Indeed, rather than measuring wall distention and assuming wall distention is proportional to blood pressure for running a non-invasive analysis per Swillens, an improved approach has been devised.

Certain embodiments of the systems, devices, and methods are capable of measuring and calculating an aortic displacement-based wave intensity (DWI) and/or a displacement-based reflected wave intensity (DRWI). A focus is on dynamic characteristics of arterial waves (pressure wave, flow/velocity wave, elastic wave, and wall displacement wave) in order to identify the deviation from a healthy or normal aorta. As such, wave pattern comparison is employed in the subject diagnoses.

Certain embodiments provide non-invasive systems, devices, and methods for the detection of AAA and other aortic aneurysms by monitoring a patient's DWI and/or DRWI at any point along the aorta or its main branches. Likewise, certain embodiments hereof provide for assessing the severity of the aortic aneurysms and/or evaluating their rupture risk by monitoring the deviation of a patient's DWI and/or DRWI at one or more points along the aorta or the aorta's main branches.

### BRIEF DESCRIPTION OF THE DRAWINGS

The figures provided herein are diagrammatic and not necessarily drawn to scale, with some components and features exaggerated and/or abstracted for clarity. Variations from the embodiments pictured are contemplated. Accordingly, depiction of aspects and elements in the figures are not intended to limit the scope of the claims, except when such intent is explicitly stated as such.

FIG. 1 is a flowchart illustrating an example embodiment of a method of aneurysm evaluation.

FIG. 2 is a diagram illustrating example embodiments of system hardware.

FIGS. 3A and 3B are example graphs of a DWI and a DRWI calculation output, respectively, of a healthy normal aorta compared to an aorta with an aneurysm at the abdomen location.

### DETAILED DESCRIPTION

The present subject matter is described in detail by way of example embodiments. It should be understood that this disclosure is not limited to these embodiments, as such may, of course, vary. It should also be understood that the terminology used herein is for the purpose of describing particular embodiments only, and is not intended to be limiting. Thus, the scope of the present disclosure should be limited only by the explicit language of the claims.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many embodiments, a method of determining a displacement-based wave intensity (DWI) or noninvasive wave intensity (ₙdI) can be employed. See, J. Feng and A. W. Khir, “Determination of Wave Speed and Wave Separation in the Arteries using Diameter and Velocity,” Journal of Biomechanics, Vol. 43(3), pp. 455-462, 2010. Wall radial displacement, D(t), is used:

dI_{D}(t)=dD(t)·dU(t) (1)

dI′_{D}(t)=(dD/dt)·(dU/dt), (2)

where dI_{D}(t) and dI′_{D}(t) are displacement based wave intensity. Consequently the DWI can be separated to forward and reflected using the below equations:

$$\begin{matrix}d {I}_{D +} = \frac{1}{4 s} {( d D + s d U )}^{2} & ( 3 ) \\ {dI}_{D -} = \frac{- 1}{4 s} {( d D - s d U )}^{2} . & ( 4 )\end{matrix}$$

Where dI_{D+} is forward DWI (DFWI), dI_{D-} is reflected DWI (DRWI), and s is the slope of a D-U loop (with D, diameter, and U, velocity) at the beginning of the cardiac cycle when reflected waves are not present. Alternative equations for the calculation of DFWI and DRWI are:

$$\begin{matrix}{}_{n} d {I}_{D +} = \frac{1}{4 D / 2 c} {( d D + \frac{D}{2 c} d U )}^{2} & ( 5 ) \\ {}_{n} d {I}_{D -} = \frac{1}{4 D / 2 c} {( d D - \frac{D}{2 c} d U )}^{2} , & ( 6 )\end{matrix}$$

where c is the wave speed.

Employing this approach, a diagnosis of an AAA and other aortic aneurysm conditions can be made by non-invasive measurement and computation of DWI and DRWI at a specific point along the aorta or its major branches and comparison of the results to baseline data for a given patent or a population sample catalogue. Deviation of DWI and/or DRWI from normal condition is thereby used to assess the severity of the AAA or other aortic aneurysm and their rupture risk.

The DWI and DRWI measurement can be either direct or indirect. The indirect measurement of DWI and DRWI is through measurement wall displacement wave and velocity (or flow) wave.

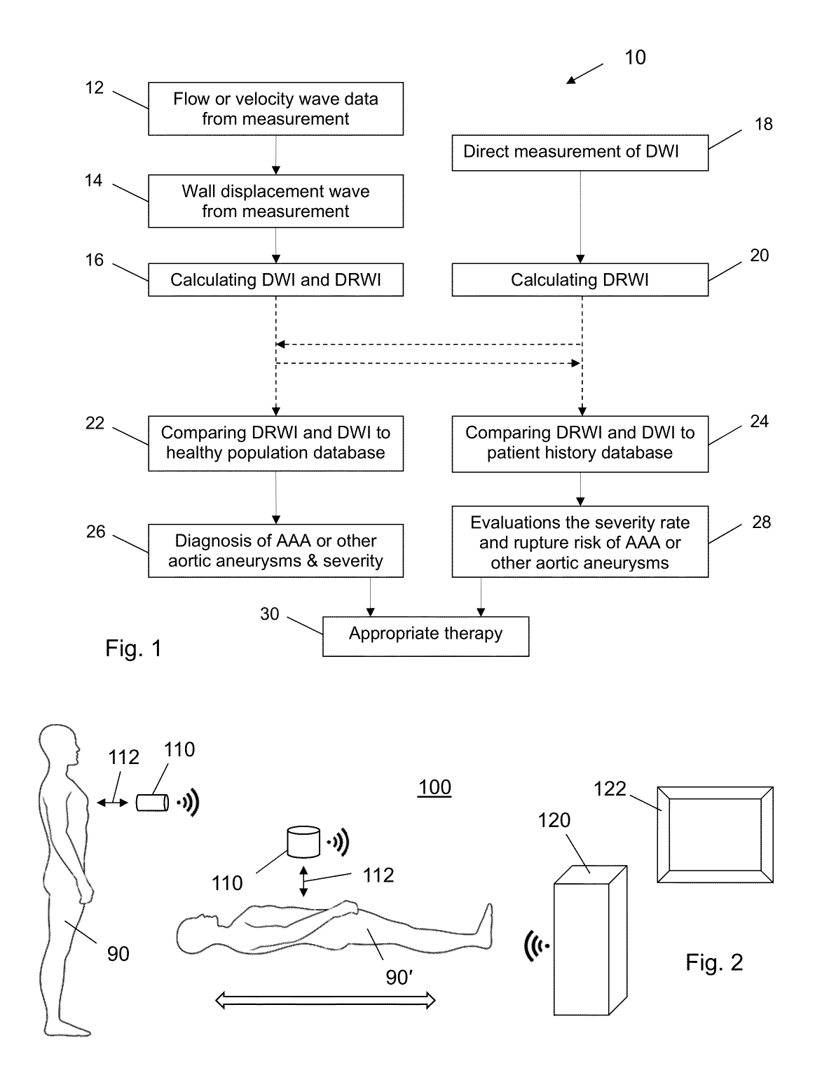

Accordingly, FIG. 1 illustrates actions in method 10 for diagnosing and/or evaluating AAAs and other types of aortic aneurysms. At 12, a flow or velocity wave is measured non-invasively at any location along the aorta or its major branches. At 14, wall displacement is measured at the same location as velocity/flow wave. Such measurement may be accomplished using ultrasound, microwave and/or other radiofrequency (RF) techniques as elaborated upon below. Using the measured data, DWI and DRWI are calculated at 16.

An alternative approach is to use devices to directly measure the DWI (e.g., with a microwave-based system or an ultrasonic-based system as in U.S. Pat. No. 6,673,020 incorporated herein by reference) at 18, and DRWI will then be calculated from the measured DWI at 20.

Depending on which process path is taken, DWI and/or DRWI may then be compared with a baseline healthy population for AAA or other aortic aneurysm diagnosis at 22. Alternatively (or additionally), DWI and/or DRWI data of the patient with an aortic aneurysm may be compared to a patient's previous condition at 24.

In the former case, the comparison against a catalogue of sample data (previously characterized) allows diagnosis of the existence and/or severity of an AAA or other aortic aneurysm at 16. In the latter case, the comparison enables patient-specific assessment of aneurysm growth, severity rate, and/or rupture risk at 28. In any case, appropriate treatment may be performed based a physician's decision in response to the subject evaluation provided. Indeed, such evaluation may include not only a quantitative and/or qualitative aneurysm of aneurysm state or status, but also suggested courses of action based on the same.

However implemented, the noted calculations of DWI and DRWI are carried out by a computer system as variously described herein. Numerous and complex mathematical operations are required as evidenced by the relevant equations, the translation of transfer signals and as represented in the subject examples. Moreover, the comparison and diagnosis or evaluation referenced above may be carried out automatically by the computer. Such comparison may be accomplished by weighing parameters as indicated in the examples below or otherwise.

A system 100 capable of carrying out the aforementioned function is illustrated in FIG. 2. Here, a computer-based system 100 with various hardware and patient-handing options is shown. A patient (alternatively referred to as a “subject”) may be scanned in a standing position 90 or in a supine position 90′ (or otherwise). A standing position may be preferable when the system scanner 110 is configured for hand-held operation. Otherwise the scanner 110′ may be larger be associated with an armature, a C-arm, a scanner “tunnel” or otherwise configured. The scanner may be moved relative to the patient to scan one or more selected areas, or the patient may be moved relative to the scanner (as indicated).

In any case, scanner 110/110′ includes an on-board transducer and electronics for sending and receiving signals 112 to perform the referenced measurements. Use of a microwave sensor (at least for measuring vessel displacement) and/or ultrasound sensors (for measuring either or both of vessel distension and blood velocity) for such purposes is well known. An example of suitable publicly-available hardware includes the GE LOGIQ Book Portable Ultrasound Machine, which technology is readily adapted to the subject methods and systems.

Alternatively, a hand-held scanner 110 incorporated in system 100 may advantageously be battery-powered so as to avoid connection to a wall socket. Whether hand-held or incorporated or in a larger entity or unity, the scanner device(s) may interface by wireless (as indicated) or wired (not shown) communication with a general purpose computer 120, optionally including display 122 to prompt user action (e.g., via a Graphical User Interface) to perform and communicate results, respectively. Otherwise, on-board processing and/or display hardware may be provided in connection with the sensor housing itself. Such options would be especially useful for a hand-held or a semi-portable device as these may be used by a patient/subject at home, during travel, and so forth.

### EXAMPLE

Numerical simulation was performed for models of each of a health normal aorta and an otherwise-identical aorta with an aneurysm in an abdominal location. Data collected for DWI calculation was taken at 6 cm away from the aortic input.

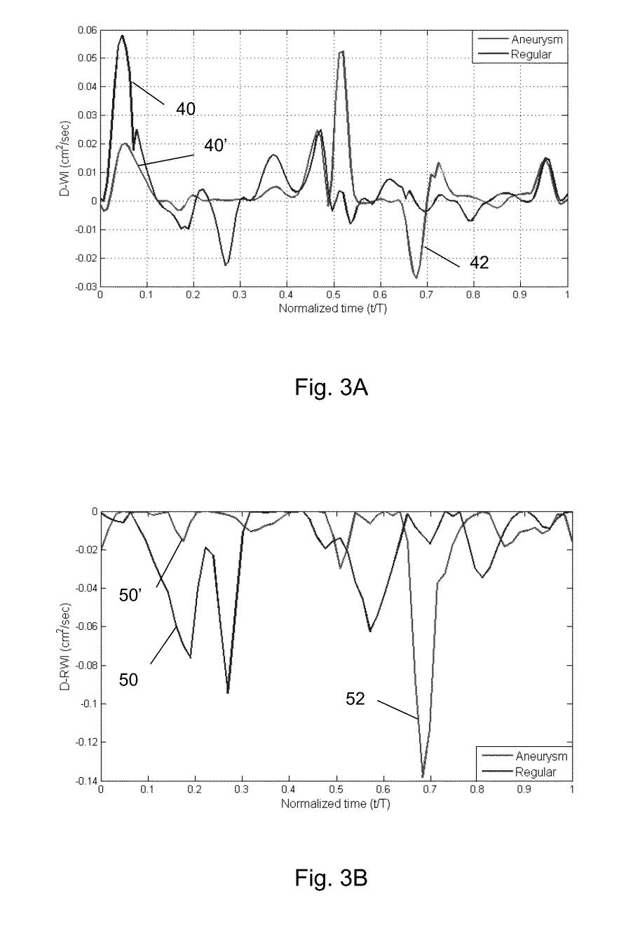

Accordingly, FIG. 3A illustrates a DWI calculation for the healthy aorta model and a DWI calculation for the aorta model with an AAA. As shown, existence of the aneurysm altered the pattern and amplitude of the peak of the DWI calculated at aortic input location. In addition, the negative part of the DWI was diminished. Comparison may be made in terms of a reduction in the magnitude of a first DWI peak 40/40′. Comparison may also (or alternatively) be made in terms of the generation or elevation of the magnitude of a DRWI peak 42 in the diastolic phase (second half of the cardiac cycle). Either one or both such indices may provide an indication and/or progression of an AAA.

Next, FIG. 3B shows a DRWI calculation for the healthy aorta and a DRWI calculation for the aorta with AAA. As illustrated, the pattern of the DRWI dramatically changed due to existence of an aneurysm in the aorta. Comparison may be made in terms of a reduction in the magnitude of a first DRWI peak 50/50′. Comparison may also (or alternatively) be made in terms of the generation or elevation of the magnitude of a DRWI peak 52 in the diastolic phase (second half of the cardiac cycle) indicates progression of AAA. Either one or both such indices may provide an indication and/or progression of an AAA.

Variations

In addition to the embodiments that been disclosed in detail above, still more are possible within the classes described and the inventors intend these to be encompassed within this Specification and claims. This disclosure is intended to be exemplary, and the claims are intended to cover any modification or alternative which might be predictable to a person having ordinary skill in the art.

Moreover, the various illustrative processes described in connection with the embodiments herein may be implemented or performed with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. The processor can be part of a computer system that also has a user interface port that communicates with a user interface, and which receives commands entered by a user, has at least one memory (e.g., hard drive or other comparable storage, and random access memory) that stores electronic information including a program that operates under control of the processor and with communication via the user interface port, and a video output that produces its output via any kind of video output format, e.g., VGA, DVI, HDMI, DisplayPort, or any other form.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein. The camera may be a digital camera of any type including those using CMOS, CCD or other digital image capture technology.

The steps of a method or algorithm described in connection with the embodiments disclosed herein may be embodied directly in hardware, in a software module executed by a processor, or in a combination of the two. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In one or more exemplary embodiments, the functions described may be implemented in hardware, software, firmware, or any combination thereof. If implemented in software, the functions may be stored on, transmitted over or resulting analysis/calculation data output as one or more instructions, code or other information on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. The memory storage can also be rotating magnetic hard disk drives, optical disk drives, or flash memory based storage drives or other such solid state, magnetic, or optical storage devices.

Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

Operations as described herein can be carried out on or over a website. The website can be operated on a server computer, or operated locally, e.g., by being downloaded to the client computer, or operated via a server farm. The website can be accessed over a mobile phone or a PDA, or on any other client. The website can use HTML code in any form, e.g., MHTML, or XML, and via any form such as cascading style sheets (“CSS”) or other.

Also, the inventors intend that only those claims which use the words “means for” are intended to be interpreted under 35 USC 112, sixth paragraph. Moreover, no limitations from the specification are intended to be read into any claims, unless those limitations are expressly included in the claims. The computers described herein may be any kind of computer, either general purpose, or some specific purpose computer such as a workstation. The programs may be written in C, or Java, Brew or any other programming language. The programs may be resident on a storage medium, e.g., magnetic or optical, e.g. the computer hard drive, a removable disk or media such as a memory stick or SD media, or other removable medium. The programs may also be run over a network, for example, with a server or other machine sending signals to the local machine, which allows the local machine to carry out the operations described herein.

Further, it is contemplated that any optional feature of the embodiment variations described may be set forth and claimed independently, or in combination with any one or more of the features described herein. Reference to a singular item, includes the possibility that there is a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

1. A system for non-invasive aortic aneurysm evaluation of a subject, comprising:
at least one sensor, the at least one sensor adapted to produce a first data set that correlates to distension of a blood vessel and to produce a second data set that correlates to blood velocity within the vessel; and
at least one processor, the at least one processor configured to convert the first data set to a measurement of blood vessel distention and the second data set to a measurement of blood velocity, to calculate from the distention and velocity measurements at least one of a displacement-based wave intensity (DWI) and a displacement-based reflected wave intensity (DRWI), and to compare at least one of the DWI and the DRWI to baseline data.

2. The system of claim 1, wherein the baseline data is from the subject.

3. The system of claim 1, wherein the baseline data is from a sample catalogue.

4. The system of claim 1, wherein the processor is further configured to output an indication of a presence of an aneurysm.

5. The system of claim 4, wherein the processor is further configured to output an indication of a severity of an aneurysm.

6. The system of claim 4, wherein the processor is further configured to output an indication of a risk of aneurysm rupture.

7. The system of claim 1, configured to calculate both the DWI and the DRWI.

8. The system of claim 7, configured to compare both the DWI and the DRWI, respectively, to baseline data.

9. The system of claim 8, configured to compare at least one of the DWI and the DRWI to both patient and sample catalogue data.

10. The system of claim 1, wherein a first positive DWI peak is compared.

11. The system of claim 1, wherein a first positive DRWI peak is compared.

12. The system of claim 1, wherein a negative DWI peak in a diastolic phase is compared.

13. The system of claim 1, wherein a negative DRWI peak in a diastolic phase is compared.

14. A method of noninvasive aortic aneurysm evaluation of a subject comprising:
calculating at least one of a displacement-based wave intensity (DWI) and a displacement-based reflected wave intensity (DRWI) from blood vessel distention and blood velocity values;
comparing at least one of the DWI and the DRWI to baseline data; and
outputting an evaluation of an aneurysm state based on the comparison.

15. The method of claim 14, further comprising:
converting a first data set to a measurement of blood vessel distention and a second data set to a measurement of blood velocity.

16. The method of claim 14, wherein the evaluation indicates aneurysm existence.

17. The method of claim 14, wherein the evaluation indicates aneurysm progression.

18. A computer readable medium having stored thereon instructions, which when executed cause one or more processors to:
calculate at least one of a displacement-based wave intensity (DWI) and a displacement-based reflected wave intensity (DRWI) from blood vessel distention and blood velocity values;
compare at least one of the DWI and the DRWI to baseline data; and
output an evaluation of aneurysm state based on the comparison.

19. The computer readable medium of claim 18, wherein the instructions further cause the one or more processors to:
convert a first data set to a measurement of blood vessel distention and a second data set to a measurement of blood velocity.

20. The computer readable medium of claim 18, wherein the evaluation indicates at least one of aneurysm existence or progression.
